(19) United States
(12) Patent Application Publication
(10) Pub. No.: US 2025/0225272 A1
(43) Pub. Date: Jul. 10, 2025

(54) AUTHENTICATION FOR A MEDICAL DEVICE

(71) Applicant: Welch Allyn, Inc., Skaneateles Falls, NY (US)

(72) Inventor: Thomas Henry Briggs, Frederick, MD (US)

(21) Appl. No.: 19/011,908

(22) Filed: Jan. 7, 2025

Related U.S. Application Data
(60) Provisional application No. 63/619,122, filed on Jan. 9, 2024.

(51) Int. Cl. G06F 21/62; A61B 90/98; G06F 21/44; G06F 21/64; G16H 40/60

(52) U.S. Cl. CPC G06F 21/6245; A61B 90/98; G06F 21/44; G06F 21/64; G16H 40/60; A61B 2562/08

(57) ABSTRACT

A medical device for securely storing information is described. The medical device includes a security device that generates one or more initial platform configuration registers corresponding to hardware information and a current operating state of the medical device. The security device generates one or more subsequent platform configuration registers corresponding to the hardware information and the current operating state when the medical device is in use after the time of manufacture. The security device compares the one or more initial platform configuration registers to the one or more subsequent platform configuration registers, and provides access to the memory when the one or more initial platform configuration registers match the one or more subsequent platform configuration registers.

### CROSS REFERENCE TO RELATED APPLICATION

This application claims the benefit of and priority to U.S. Provisional Patent Application No. 63/619,122, filed on 9 Jan. 2024, the disclosure of which is hereby incorporated by reference in its entirety.

### BACKGROUND

Caregivers typically use various types of healthcare equipment to provide healthcare to a patient in a clinical care environment. In certain examples, healthcare equipment can include a memory storage device that at least temporarily stores private health information (PHI) of the patient. PHI may include, for example, demographic and medical history information, health monitoring data from one or more monitoring devices, lab results, insurance information, prior communications between the patient and caregivers, and emergency contact information.

### SUMMARY

In general terms, the present disclosure relates to a medical device that includes at least one security device. In one possible configuration, the medical device exhibits a technical effect by utilizing at least one security device to control access to a memory based on a comparison of one or more initial platform configuration registers to one or more subsequent platform configuration registers. Various aspects are described in this disclosure, which include, but are not limited to, the following aspects.

One aspect relates to a medical device comprising: at least one processor; a memory storing data and firmware including hardware information and a current operating state; and at least one security device integrated into the medical device, the at least one security device including: at least one security device processor; and a security device memory storing instructions which, when executed by the at least one security device processor, cause the at least one security device processor to: generate one or more initial platform configuration registers corresponding to the hardware information and the current operating state at a time of manufacture; generate one or more subsequent platform configuration registers corresponding to the hardware information and the current operating state when the medical device is in use after the time of manufacture; compare the one or more initial platform configuration registers to the one or more subsequent platform configuration registers; and provide access to the memory when the one or more initial platform configuration registers match the one or more subsequent platform configuration registers.

Another aspect relates to a method for storing information within a medical device, the method comprising: integrating a security device into the medical device at a time of manufacture; generating one or more initial platform configuration registers that correspond to medical device hardware information and a current operating state of the medical device at the time of manufacture; and storing the one or more initial platform configuration registers within the security device.

Another aspect relates to a security system for integration into a medical device; the security system comprising: at least one security device processor; and a security device memory storing instructions which, when executed by the at least one security device processor, cause the at least one security device processor to: generate one or more initial platform configuration registers corresponding to hardware information and a current operating state of the medical device at a time of manufacture; generate one or more subsequent platform configuration registers corresponding to the hardware information and the current operating state when the medical device in use after the time of manufacture; compare the initial platform configuration registers to the subsequent platform configuration registers; and provide access to the memory when the one or more initial platform configuration registers match the one or more subsequent platform configuration registers.

Another aspect relates to a method for accessing information stored within a medical device, the method comprising: receiving an input requesting access to a memory within the medical device; generating one or more platform configuration registers corresponding to hardware information and a current operating state when the medical device is in use after a time of manufacture; comparing the one or more platform configuration registers to one or more initial platform configuration registers stored within a security device memory, wherein the one or more initial platform configuration registers correspond to the hardware information and the current operating state at the time of manufacture; and providing access to the memory when the one or more initial platform configuration registers match the one or more subsequent platform configuration registers.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for collecting physiological parameter measurements from patients in a clinical care environment.

FIG. 2 illustrates an example of a medical device of the system of FIG. 1.

FIG. 3 schematically illustrates an example of the medical device of FIG. 2.

FIG. 4 schematically illustrates examples of authenticity identifiers used by a security device to determine the authenticity of the medical device of FIG. 3.

FIG. 5 schematically illustrates an example of a method of accessing information within the medical device of FIG. 3.

FIG. 6 schematically illustrates an example of a subroutine of the method of FIG. 5 for measuring various components of the medical device of FIG. 3 to generate platform configuration registers.

FIG. 7 schematically illustrates an example of a method of determining a policy for protecting private information within the medical device of FIG. 3 by blocking access to certain users based on the determined policy.

### DETAILED DESCRIPTION

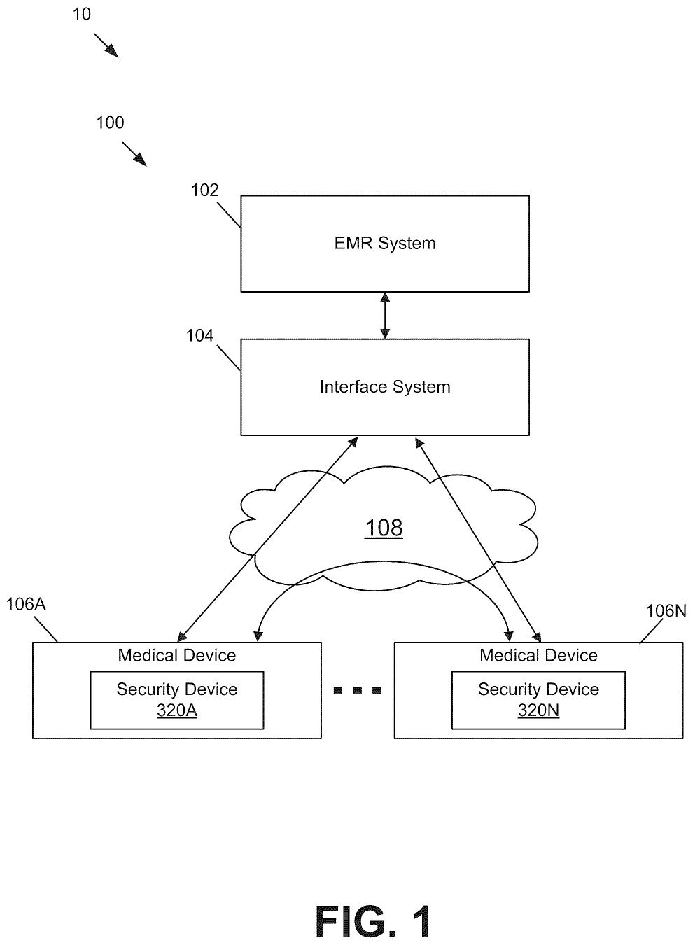

FIG. 1 schematically illustrates an example of a system 100 for collecting physiological parameter measurements from patients in a clinical care environment 10. An example of the clinical care environment can include a healthcare facility such as a hospital, a surgical center, a nursing home, a long-term care facility, or similar type of facility. As shown in FIG. 1, the system 100 includes an Electronic Medical Records (EMR) system 102, an interface system 104, medical devices 106A-106N, security devices 320A-320N, and a network 108. As used herein, a medical device 106 includes one or more instruments used to collect data pertaining to the health of a patient.

The network 108 is a communications network that facilitates data communication between the medical devices 106A-106N, and between the medical devices 106A-106N and the interface system 104. The network 108 can include computing devices and links between the computing devices. The computing devices in the network 108 use the links to enable data communication among the computing devices in the network. The network 108 can include routers, switches, mobile access points, bridges, hubs, intrusion detection devices, storage devices, standalone server devices, blade server devices, sensors, desktop computers, firewall devices, laptop computers, tablet computers, handheld computers, smartphones, and other types of computing devices. In various embodiments, the network 108 includes various types of links. For example, the network 108 can include wired and/or wireless links. In some embodiments, the network 108 is implemented at various scales. For example, the network 108 can be implemented as one or more local area networks (LANs), metropolitan area networks, subnets, wide area networks (such as the Internet), or can be implemented at another scale.

The EMR system 102 is a computing system that provides storage, retrieval, and manipulation of electronic medical records. A computing system can include one or more computing devices. A computing device is a physical, tangible device that processes data. Example types of computing devices include personal computers, standalone server computers, blade server computers, mainframe computers, laptop computers, tablet computers, handheld computers, smartphones, and other types of electronic devices that process data. The electronic medical records stored in the EMR system 102 can include protected health information (PHI).

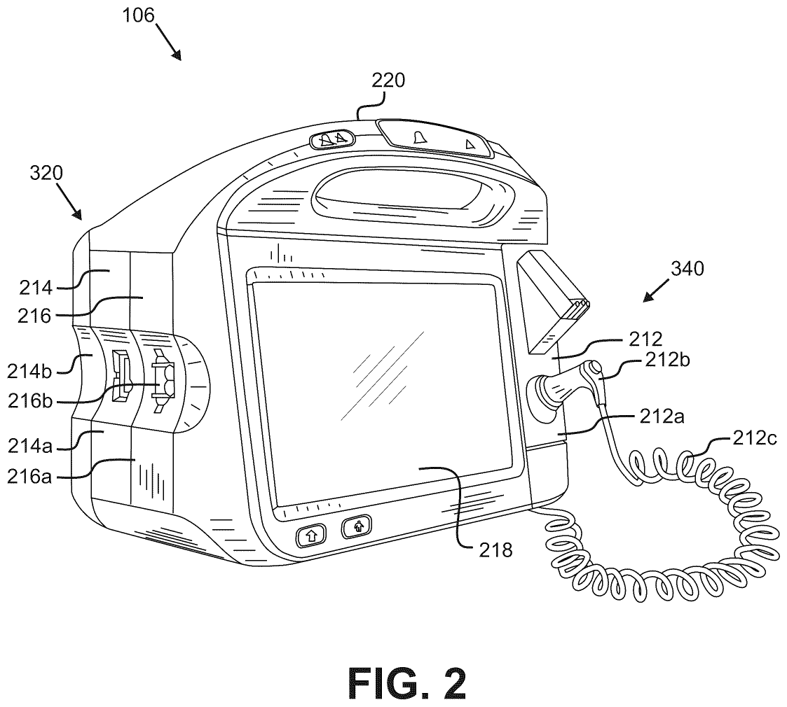

Each of the medical devices 106 is a computing device. The medical devices 106 can provide various types of functionalities. For example, the medical devices 106 can include a physiological parameter monitoring platform or spot monitor, such as the one illustrated in FIG. 2. In such examples, the physiological parameter monitoring platform or spot monitor can be used by a clinician to measure and/or monitor physiological parameters of one or more patients, and to display representations of the measured physiological parameters.

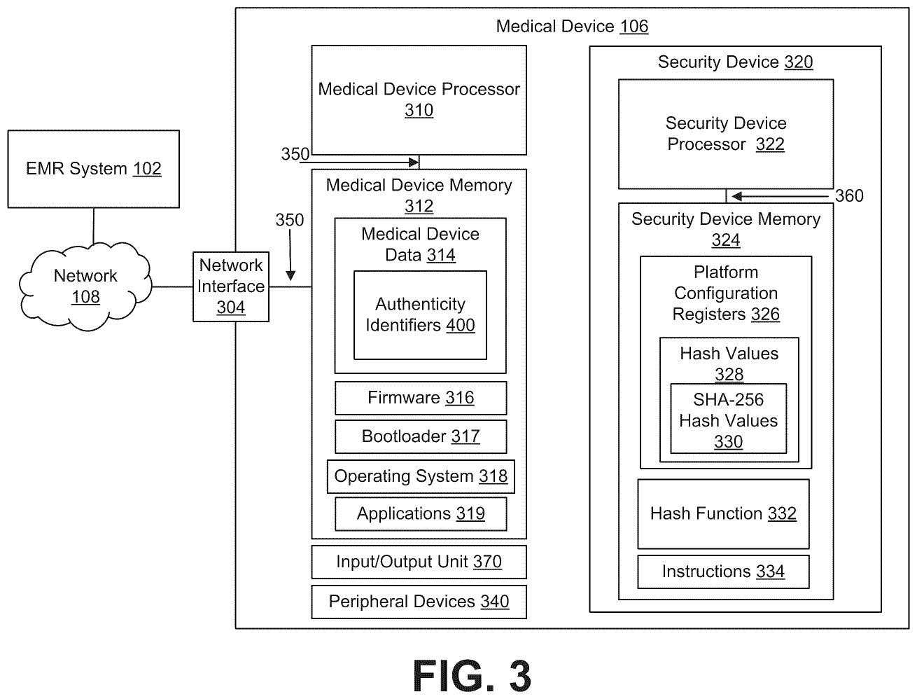

One or more security devices 320 are integrated into each of the medical devices 106. The one or more security devices 320 may include software, hardware, or a combination thereof for securing access to PHI stored in a memory of the medical device 106 or in the EMR system 102, which is accessible to the medical devices 106 via the network 108. The security devices 320 may include one or more of a trusted platform module (TPM), a hardware security module (HSM), firewalls, intrusion detection and prevention systems, encryption devices, virtual private network devices, and other access control systems. The one or more security devices 320 include a security device processor 322 and a security device memory 324 configured to store and execute instructions for verifying the authenticity of the medical devices 106 (as illustrated and described in further detail in FIG. 3). The authenticity of the medical device 106 is used herein to describe when the medical device 106 is in a trusted state (i.e., when components within the medical device 106 are determined to be genuine without any tampering that could adversely affect the protection of information within a memory of the medical device 106).

In certain examples, the medical devices 106 can include IoT (Internet of Things) functionality via connection to the network 108. The medical devices 106 are equipped with sensors, software, and other technologies to collect and transmit health-related data to other systems and devices such as the EMR system 102. Connections to the EMR system 102 and to other IoT devices through the network 108 may enable remote monitoring of a patient and real- time communication between a medical device 106 and a clinician.

The medical devices 106 can include one or more peripheral devices operatively coupled to the medical devices 106. Examples of peripheral devices include, without limitation, a thermometry sensor module, a blood pressure sensor module, ECG or EKG leads for connection with an electrocardiogram monitor, a photoplethysmogram sensor module for measuring blood oxygen saturation and pulse, and other peripheral devices. The medical devices 106 can further include other types of devices for measuring physiological parameters such as hospital beds. The medical devices 106 can communicate with each other through the network 108.

The interface system 104 is a computing system that acts as an interface between the EMR system 102 and the medical devices 106. In some embodiments, the interface system 104 includes Connex® Vitals Management Software from Hillrom of Batesville, Indiana.

As an example, the interface system 104 provides a single software interface for each of the medical devices 106 such that the medical devices can send requests to the software interface provided by the interface system 104. When the interface system 104 receives a request from one of the medical devices 106, the interface system 104 translates the request into a request that is compatible with the software interface provided by the EMR system 102. The interface system 104 then provides the translated request to the EMR system 102. When the interface system 104 receives a response from the EMR system 102, the interface system 104 translates the response into a format understood by the medical devices 106 and forwards the translated response to one of the medical devices 106. In this manner, the interface system 104 allows use of the medical devices 106 in various types of EMR systems 102.

The medical devices 106 can send various types of data to the interface system 104 for storage in the EMR system 102 and can receive various types of data from the EMR system 102 through the interface system 104. For example, a medical device 106 can send physiological parameter measurements and clinical observations to the interface system 104 for storage in the EMR system 102. In further examples, a medical device 106 can retrieve past physiological parameter measurements, clinical observations, lab results, scans, and other patient health information from the EMR system 102 through the interface system 104.

FIG. 2 illustrates an example of a medical device 106 of the system 100. In this example, the medical device 106 is a physiological parameter monitoring platform or spot monitor. The medical device 106 can include one or more sensor modules. Each of the sensor modules is configured to measure one or more physiological parameters of a patient.

In the illustrative example shown in FIG. 2, the medical device 106 includes one or more peripheral devices 340, which are described in further detail with respect to FIG. 3. In the example shown, the peripheral devices 340 include a thermometry sensor module 212 that is accessible from a front side of the device, and a photoplethysmogram sensor module 214 and a non-invasive blood pressure (NIBP) sensor module 216 that are accessible from a left-hand side of the device. As used herein, a “module” is a combination of physical structure which resides in the medical device 106 and peripheral components that attach to and reside outside of the medical device 106. The medical device 106 can include additional sensor modules for receiving additional physiological parameter measurements, including ECG or EKG measurements.

The thermometry sensor module 212 is designed to receive body temperature measurements of a patient. As an illustrative example, the thermometry sensor module 212 includes a front panel 212a that has an outer surface accessible from the front side of the medical device 106. The front panel 212a provides access to a receptacle for storing a removable probe, also referred to as a temperature probe, attached to a probe handle 212b. The temperature probe and the probe handle 212b are tethered to the thermometry sensor module 212 via an insulated conductor 212c. The temperature probe is configured to make physical contact with the patient in order to sense the body temperature of the patient.

The photoplethysmogram sensor module 214 can be used to measure blood oxygen saturation and pulse. Also, the photoplethysmogram sensor module 214 can be used to measure respiration rate based on changes in a plethysmography waveform. The photoplethysmogram sensor module 214 includes a front panel 214a having a connector port 214b that enables a connection between a pulse oximeter and the photoplethysmogram sensor module 214 residing inside the medical device 106. The pulse oximeter resides externally and is configured to interoperate with the photoplethysmogram sensor module 214 when connected to the photoplethysmogram sensor module 214 via the connector port 214b. The pulse oximeter can include a clip that attaches to an appendage (e.g., finger) of the patient. The clip includes an infrared light transmitter, and a sensor that detects and measures blood oxygen saturation and pulse rate based on transmission of the infrared light through the patient's appendage.

The NIBP sensor module 216 can be used to measure blood pressure of the patient. The NIBP sensor module 216 includes a front panel 216a having a connector port 216b enabling a connection between one or more peripheral NIBP components and the NIBP sensor module 216 residing inside the medical device 106. The peripheral NIBP components reside externally and are configured to interoperate with the NIBP sensor module 216 when connected to the NIBP sensor module 216 via the connector port 216b. The peripheral NIBP components can include an inflatable cuff that attaches to an appendage of the patient, such as an upper arm. The inflatable cuff is used to measure systolic and diastolic blood pressure of the patient, mean arterial pressure (MAP) of the patient, and pulse rate of blood flowing within the patient.

As shown in FIG. 2, a front side of the medical device 106 includes a display screen 218. In this example, the display screen 218 is a built-in display. The display screen 218 can display graphical representations of the physiological parameter measurements received from the various sensor modules of the medical device 106 including the thermometry sensor module 212, the photoplethysmogram sensor module 214, and the NIBP sensor module 216. The display screen 218 can further display graphical representations of additional physiological parameter measurements and clinical observations from additional sources such as from other medical devices and the EMR system 102 via connections through the network 108. In some examples, the display screen 218 is a touchscreen that receives manual inputs from a user of the medical device 106. Also, the medical device 106 can include one or more handles 220 on its housing that enable the medical device 106 to be carried by hand by the user.

The medical device 106 includes the one or more security devices 320. The one or more security devices 320 operate to establish a trusted relationship between the medical device 106, an operating system of the medical device, and one or more applications used to collect patient health data. This process is described in further detail herein with respect to FIGS. 3-5.

FIG. 3 schematically illustrates an example of the medical device 106. As shown in the example provided in FIG. 3, the medical device 106 includes one or more security devices 320, one or more peripheral devices 340, and a network interface 304 for communicating with the EMR System 102 via the network 108.

The medical device 106 collects, retrieves, and stores protected health information (PHI). The security device 320 provides a secure environment for storing sensitive information such as the PHI, and for evaluating the authenticity of the medical device 106.

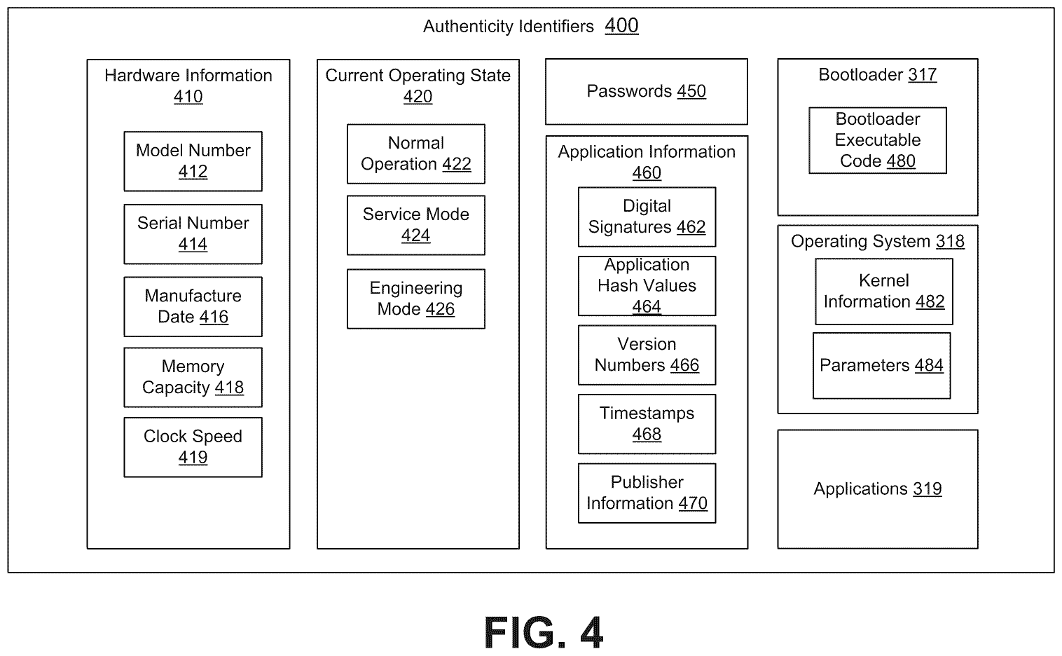

The medical device 106 includes a medical device processor 310 and the security device 320 includes a security device processor 322. The medical device processor 310 has a processing power (i.e., the medical device processor clock speed 419 as illustrated and described in further detail with respect to FIG. 4). The medical device processor 310 is connected to the medical device memory 312 through a medical device system bus 350. The security device processor 322 is connected to the security device memory 324 through a security device system bus 360. The medical device memory 312 and the security device memory 324 can each include any form of computer-readable data storage media.

It should be appreciated by those skilled in the art that computer-readable data storage media can be any available non-transitory, physical device or article of manufacture from which the medical device 106 can read data and/or instructions. The computer-readable storage media can be comprised of entirely non-transitory media.

Computer-readable data storage media include volatile and non-volatile, removable, and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device. Furthermore, the RAM may include volatile RAM (VRAM) and non-volatile RAM (NVRAM).

The medical device memory 312 stores medical device data 314, firmware 316, an operating system 318, and software applications 319 to control the functionality of the medical device 106. The medical device data 314 can include patient medical data collected from one or more of the sensor modules, patient biographic information, medical device settings and configurations, device usage logs, and other data. Furthermore, the medical device data 314 includes authenticity identifiers 400 to be used in the processes described herein to verify the authenticity of the medical device 106. The authenticity identifiers 400 are illustrated and described in further detail with respect to FIG. 4.

The firmware 316 is stored in non-volatile memory within the medical device memory 312. The firmware 316 includes instructions and code that dictate how the medical device 106 operates in a current operating state 420 (as discussed in greater detail with respect to FIG. 4). The firmware 316 performs several functions, such as booting the operating system 318, managing one or more peripheral devices 340, and executing instructions that govern hardware components within the medical devices 106. In certain examples, the firmware 316 includes one or more embedded web servers. The embedded web servers may be included in the medical device 106 to allow users to access settings, view one or more statuses of components within the medical device 106, or control the medical device 106 through a web browser.

The operating system 318 manages and controls hardware resources within the medical devices 106 and enables the execution of the software applications 319. Furthermore, the operating system 318 organizes and manages files stored within the medical device memory 312, communicates with one or more peripheral devices 340, and manages network connections to communicate with other devices and systems via the network 108.

The firmware 316 functions with the operating system 318 and the software applications 319 to perform the functions of the medical device 106. When the medical device 106 is powered on, the firmware 316 initializes and configures hardware components within the medical device 106 such as the medical device processor 310 and the medical device memory 312, checks the system's integrity, and loads a bootloader 317 from the medical device memory 312 to initiate a boot processor. The bootloader 317 then loads an operating system kernel and allows the operating system kernel to take control of the system to perform hardware checks, initialize other system components, and establish a secure environment for the operating system 318 to run. The operating system kernel boots other system components (e.g., the file system, device drivers, network interfaces, and the like) to initialize the operating system 318. Control is handed to a user once the operating system 318 is fully initialized. The user may then interact with the operating system by loading the software applications 319 to perform the functionalities of the medical device 106. In certain examples, the software applications 319 are configured to track a current operating state 420 of the software applications 319 and other hardware information 410 (as illustrated and described in further detail with respect to FIG. 4).

The medical device 106 includes an input/output unit 370 for receiving and processing inputs and outputs from a number of peripheral devices 340. Examples of peripheral devices can include, without limitation, the thermometry sensor module 212, the NIBP sensor module 216, the ECG or EKG leads, the photoplethysmogram sensor module 214 for measuring blood oxygen saturation and pulse, and other peripheral devices.

The medical device 106 operates in a networked environment using logical connections to the other medical devices and other electronic devices through the network 108. The medical device 106 connects to the network 108 through a network interface 304 connected to the medical device system bus 350. The network interface 304 can also connect to additional types of communications networks and devices, including through Bluetooth, Wi-Fi, and cellular telecommunications networks including 4G and 5G networks. The network interface 304 may also connect the medical device 106 to the EMR system 102.

The medical device memory 312 can store software instructions and data. The software instructions can include an operating system 318 and firmware 316 suitable for controlling the operation of the medical device 106. The security device memory 324 can store software instructions 334, which when executed by the security device processor 322, cause the security device 320 to provide the functionality described herein.

In certain examples, the security device 320 is a trusted platform module (TPM) integrated into the medical device 106 to enhance the security features of the medical device 106. The TPM stores one or more hash functions 332 that are configured to perform one or more cryptographic operations to generate one or more hash values 328, which can include, for example, SHA-256 hash values 330 generated from a SHA-256 hash function. The hash values 328 are stored in one or more platform configuration registers (PCRs) 326.

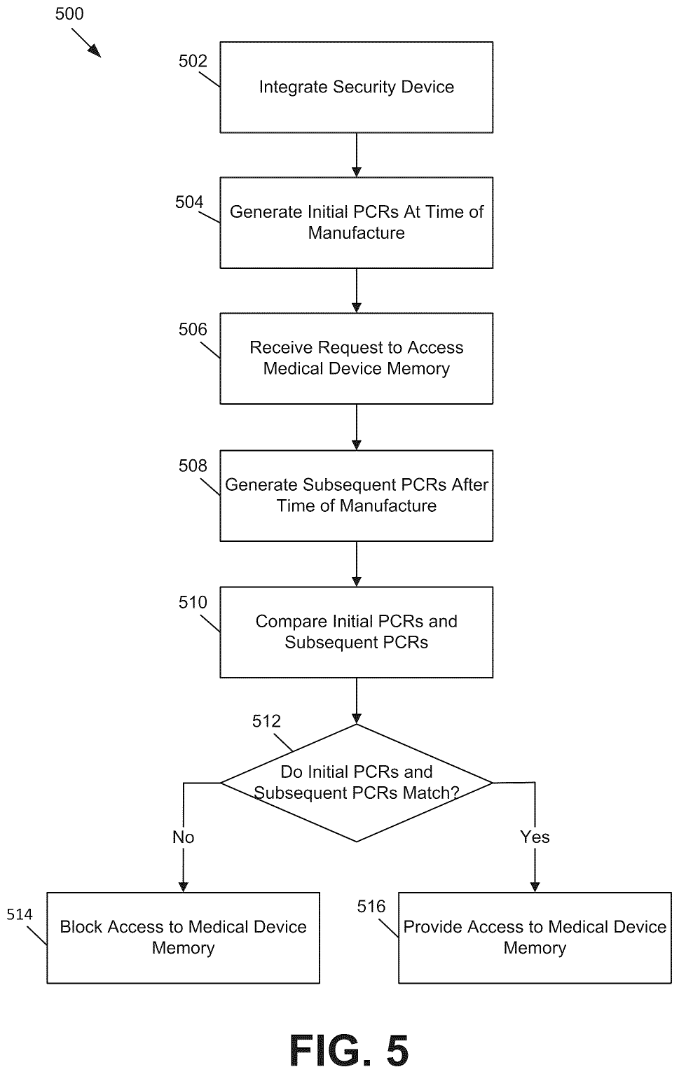

The one or more PCRs 326 include an initial platform configuration register storing one or more hash values 328 corresponding to features of the medical device 106 measured at the time of manufacture. The one or more PCRs 326 further include one or more subsequent platform configuration registers storing one or more hash values 328 corresponding to features of the medical device 106 measured at one or more times after the time of manufacture. The initial platform configuration register and the one or more subsequent platform configuration registers may be compared to verify the authenticity of various components within the medical device 106, which is described in further detail with respect to FIGS. 4-5.

The hash function 332 compares the hash values 328 stored in the initial platform configuration register and the one or more subsequent platform configuration registers. The hash function 332 provides access to the medical device memory 312 when the hash values 328 stored within the initial platform configuration register and the one or more subsequent platform configuration registers match. A process by which access is provided to the medical device memory 312 is illustrated and described in further detail with respect to FIG. 5.

At the time of manufacture, the medical device 106 is in a trusted state, meaning the medical device 106 is considered to be secure, trustworthy, and operating in a manner consistent with its intended configuration. When the medical device 106 is used within a clinical care environment 10, it may deviate from the trusted state when one or more users manipulate the hardware or software associated with the medical device 106. In certain non-limiting examples, the medical device 106 may no longer be trustworthy (i.e., enter a non-trusted state) when the authenticity of the medical device 106 has not been verified within a defined time limit, when unauthorized software or malware is installed on the medical device 106, when unauthorized modifications are made to the firmware 316, when modifications are made to various system files, when security updates are not performed, when a network attack is performed on the network 108, when the boot process is compromised, and the like. It is desirable to ensure the medical device 106 is in a trustworthy state prior to allowing access into the medical device memory 312 to maintain the integrity of PHI, remain compliant with various laws and regulations (such as the Health Insurance Portability and Accountability Act (HIPAA) in the United States), and prevent fraudulent activity such as identity theft and financial frauds. A process by which the medical device 106 verifies it is in a trusted state is illustrated and described in further detail with respect to FIG. 5.

FIG. 4 schematically illustrates examples of authenticity identifiers 400 used by the security device 320 to determine the authenticity of the medical devices 106. The authenticity identifiers 400 include hardware information 410, a current operating state 420 of the medical device 106, one or more passwords 450, and application information 460.

The security device 320 is configured to verify the authenticity of the medical device 106 by using the one or more hash functions 332 that apply a series of mathematical operations to transform input data (e.g., one or more authenticity identifiers 400) into an output that is unique to the input data. The one or more hash functions 332 generate one or more hash values 328 relating to the authenticity identifiers 400 at the time of manufacture and after the time of manufacture. The security device 320 compares the one or more hash values 328 generated at the time of manufacture to the one more hash values 328 generated after the time of manufacture to determine whether there is a match. When the one or more hash values 328 match, the medical device 106 is determined to be in a trusted state, and the security device 320 allows access to the medical device memory 312.

As used herein, generating one or more platform configurations at a time of manufacture refers to generating of one or more hash values at the time of manufacture and storing the one or more hash values within an initial platform configuration register. Furthermore, generating one or more platform configurations after the time of manufacture refers to generating of one or more hash values after the time of manufacture and storing the one or more hash values within a subsequent platform configuration register. A process for verifying the authenticity of the medical device 106 by comparing the initial platform configuration register with one or more subsequent platform configuration registers is illustrated and described in further detail with respect to FIG. 5.

In certain examples, the hardware information 410 includes at least one of a model number 412, a serial number 414, a date of manufacture 416, a memory capacity 418, and a medical device processor clock speed 419. The model number 412 is a unique identifier assigned to a specific model or version of the medical device 106. The model number 412 allows the medical device 106 associated with that model number 412 to be identified, tracked, and managed within the clinical care environment 10. In certain examples, the model number 412 includes alphanumeric characters, codes, or designs that convey information pertaining to the features of the medical device 106 or its intended use.

The serial number 414 is a unique identifier associated with an individual unit of a medical device 106. In contrast to the model number 412, the serial number allows a single instance of the medical device 106 to be identified rather than a type or version of the medical device 106, which may contain a plurality of instances.

The date of manufacture 416 relates to a date when a single instance of the medical device 106 was produced or assembled. The date of manufacture 416 is useful in distinguishing between various instances of the medical device 106 because it allows for the determination of the age of a various instance of the medical device and an understanding of where the device is in its lifecycle.

The memory capacity 418 of the medical device 106 relates to the storage space within the medical device memory 312. The memory capacity 418 is useful in distinguishing between various models or instances of the medical device 106 where different memory capacities are required within the medical device memory 312. Memory capacity is measured in bytes (B) and often expressed in megabytes (MB), gigabytes (GB), or terabytes (TB). In certain examples, hash values 328 of various measurements of the medical device memory 312 are calculated. In a non-limiting example, the bootloader 317 can measure a random-access memory (RAM) capacity and an embedded multimedia card (“eMMC”) storage capacity.

The medical device processor clock speed 419 is a measurement of the medical device processor 310 (e.g., a central processing unit CPU) relating to the frequency at which the medical device processor 310 executes instructions stored within the medical device memory 312 and performs operations. The medical device processor clock speed 419 is measured in hertz (Hz) and often expressed in megahertz (MHz) or gigahertz (GHz). The medical device processor clock speed 419 of the medical device processor 310 is useful in distinguishing between various models or instances of the medical device 106 where differing amounts of processing power are required to perform the functionalities of the medical device 106. Furthermore, hash values 328 of various measurements of the medical device processor 310 may include physical properties of the medical device processor 310 such as manufacturer, model number, and serial number.

In certain examples, the current operating state 420 includes at least one of a normal operation mode 422, a service mode 424, and an engineering mode 426. In the normal operation mode 422, the medical device 106 is configured to collect patient data. In the service mode, the medical device 106 is configured to be upgraded, calibrated, or otherwise repaired by technicians. In the engineering mode, the medical device 106 is configured to be tested or optimized by a developer, engineer, or researcher. The medical device 106 is configured to track the current operating state 420 during use and adjust access to various functionalities of the medical device depending on the current operating state 420.

In certain examples, one or more functionalities of the medical device 106 are blocked based on the current operating state 420. In a non-limiting example, the security device 320 may block access to private health information (PHI) stored within the medical device 106 when the current operating state is in the service mode or the engineering mode. In another non-limiting example, the security device 320 may block access to manufacturer-provided service certificates (i.e., documentation of service provided to the medical device 106) when the current operating state is in the normal operation mode or the engineering mode. Access to the PHI is blocked to individuals in the normal operation mode 422 when the security device 320 determines one or more components within the medical device memory 312 are not authentic.

In certain examples, one or more functionalities of the medical device 106 may be blocked using a public key infrastructure (PKI) for providing access to the service software applications installed on the medical device 106. A certificate authority (CA) can issue signed certificates for various entities and individuals to service the medical device 106. The CA can be managed by the manufacturer of the medical device 106.

In this example, the CA issues a first type of signed certificate to a service department and a second type of signed certificate to an engineering department. The service department and the engineering department can be departments within the manufacturer of the medical device 106. The service department and the engineering department can distribute their respective signed certificates to their individual members such as service personnel and testing engineers.

Each of the different types of signed certificates can provide a different level of access to the firmware 316, the operating system 318, and the software applications 319 based on the credentials of a user. In further examples, the signed certificates can define a date range during which the signed certificates are valid, and after which, the signed certificates are expired. This can be done to verify that the signed certificates remain effective and/or authorized by the CA. The signed certificates when stored on the security device memory 324 allow the medical device 106 to identify, authenticate, and authorize the various users without requiring a network connection such as to the internet.

As an illustrative example, the first signed certificate provides the service personnel access to a standard set of service software applications for servicing the medical device 106, while the second type of signed certificate provides a higher level of access for the testing engineers. For example, the higher level of access can allow the testing engineers TE to perform testing and debugging of the 316, which is not allowed to be performed by the service personnel. Additional examples how the signed certificates can provide different levels of access to the firmware 316 are possible.

In certain examples, one or more passwords 450 are used to verify the authenticity of a user that is attempting to access information stored within the medical device memory 312. In a non-limiting example, one or more passwords 450 are stored within the security device memory 324 at the time of manufacture or at another time after manufacture when the medical device 106 is determined to be in a trusted state. After the time of manufacture, the security device 320 requests an input from one or more users to verify the one or more passwords 450. The security device 320 receives the input from the user containing one or more passwords 450 and provides access to the medical device memory 312 when the one or more stored passwords 450 match the input from the one or more users. In certain examples, the one or more passwords 450 are used alone or in conjunction with other verification methods described herein to determine the authenticity of a user or medical device 106. In other examples, the one or more passwords 450 are not used to verify the authenticity of the user.

The application information 460 includes information that is used to authenticate the software applications 319. This information can include, by non-limiting example, digital signatures 462 from an application provider (as described below), application hash values 464 pertaining to the software applications 319, version numbers 466 of the application, timestamps 468 (indications of when the software applications 319 were signed by the application creator), and other publisher information 470. It is desirable to authenticate software used to operate the medical devices 106 to ensure the medical device 106 is in a trusted state.

In some examples, digital signatures may be used to verify the authenticity of the software applications 319. The digital signature may include a unique digital fingerprint (e.g., a hash value) generated from the contents of the software applications 319 that is encrypted using a private key from the original application creator. Recipients of the software applications 319 can then verify the application is authentic by decrypting the private key with the application provider's public key and comparing the result with a locally computed hash value of the software application 319.

FIG. 5 schematically illustrates an example of a method 500 of accessing information within the medical device 106. The method 500 includes a step 502 of integrating the security device 320 into the medical device 106 at the time of manufacture. In certain examples, the security device 320 is a trusted platform module (TPM) that is manufactured separately from the medical device 106 and embedded onto a motherboard of the medical device 106 during the manufacturing process. The security device 320 is connected to the medical device system bus 350 to allow for communication between the security device 320 and the medical device 106.

In some examples, the functionality of the security device 320 is integrated into the firmware 316 of the medical device 106 to allow the security device 320 to be initialized during the boot process. The manufacturer may install drivers to allow the operating system 318 to interact with the security device 320.

The method 500 includes a step 504 of generating initial platform configuration registers at the time of manufacture. Generating initial platform configuration registers (PCRs) includes using one or more hash functions 332 to generate hash values 328 and storing the hash values 328 in one or more initial platform configuration registers (PCRs). An illustrative example of a hash function 332 that can be used includes the SHA-256 hash function.

The hash values 328 are measurements of unique features of the medical device 106. The hash values 328 exhibit an “avalanche effect,” where small differences in input data (e.g., a measurement of a component within the medical device 106) result in a significantly different outputted hash value when comparing a hash value of the original measurement with a hash value of an altered measurement. Furthermore, the hash values 328 exhibit a “trap-door property,” where it is possible to compute a hash value 328 from an input measurement using a hash function 332, but is effectively impossible to make the reverse computation of determining the original input measurement using the hash value computed from the input measurement.

In certain examples, the security device 320 generates hash values 328 of unique characteristics of the bootloader 317, the operating system 318, the software applications 319, and other authenticity identifiers 400. During boot up, the bootloader 317 measures various characteristics of the medical device processor 310 and the medical device memory 312. These measurements may include, by non-limiting example, at least one of the manufacturer of the medical device processor, the medical device processor clock speed, a model number and serial number for each component, and a storage capacity of the medical device memory 312. The bootloader 317 may include stored values encoded into bootloader executable code 480 for the bootloader 317 stored within the medical device memory 312.

Before the bootloader 317 transfers control to the operating system 318 (as discussed above with reference to FIG. 3), one or more metrics of the operating system 318 are measured and hash values 328 of the operating system 318 are calculated from the measurements. In certain examples, the one or more metrics of the operating system can include kernel information 482 relating to the operating system kernel and additional parameters 484 passed to the operating system kernel during the bootup process (e.g., a kernel version, a kernel command line, or any memory-related parameters). When the operating system 318 takes control during the bootup process, hash values 328 of the software applications 319 are measured. These include timestamps (indications of when the software applications 319 were signed by the application creator), version numbers of the software applications 319, license keys, watermarks, and other methods of authenticating software as illustrated and described above with respect to FIG. 4.

In certain examples, the one or more hash functions 332 also generate hash values 328 pertaining to other authenticity identifiers 400 at the time of manufacture. This can include, by non-limiting example, the current operating state 420 of the medical device 106, or passwords 450 inputted into the security device memory 324. The authenticity identifiers 400 are illustrated and described in further detail with respect to FIG. 4.

The method 500 includes a step 506 of receiving a request to access information stored in the medical device memory 312. The information may include, for example, PHI. In certain examples, the request to access non-volatile memory, such as PHI, can come during the bootup process as the operating system 318 is initialized. It is desirable for the security device 320 to verify the authenticity of hardware and software within the medical device 106 prior to providing access to non-volatile memory stored within the medical device memory 312.

The method 500 includes a step 508 of generating subsequent platform configuration registers after the time of manufacture. In certain examples, step 508 includes similar processes performed in step 504. In a non-limiting example, one or more initial platform configuration registers are generated at the time of manufacture that include stored hash values 328 corresponding to the hardware information 410 and the current operating state 420 of the medical device 106. Furthermore, the one or more subsequent platform configuration registers include stored hash values corresponding to the hardware information 410 and the current operating state 420 when the medical device is in use after the time of manufacture.

The method 500 includes a step 510 of comparing the initial platform configuration register to the one or more subsequent platform configuration registers to determine whether hash values 328 stored within the PCRs 326 match. When the security device 320 determines in a step 512 that the hash values 328 stored within the initial platform configuration register and the one or more subsequent platform configuration registers match, the security device 320 proceeds to a step 516 of providing access to the medical device memory 312. When the security device 320 determines the hash values 328 stored within the initial platform configuration register and the one or more subsequent platform configuration registers do not match, the security device 320 proceeds to a step 514 of blocking access to the medical device memory 312.

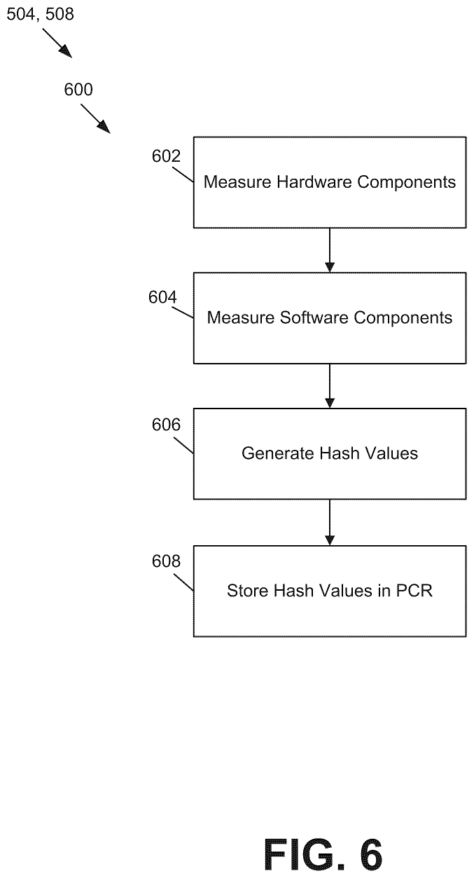

FIG. 6 schematically illustrates an example of a subroutine 600 of the method 500 for measuring various components of the medical device 106 to generate the PCRs 326. The subroutine 600 can be performed during step 504 of the method 500 to generate the initial PCRs at the time of manufacture and/or can be performed during step 508 of the method 500 to generate the subsequent PCRs after the time of manufacture.

The subroutine 600 includes a step 602 of measuring one or more hardware components of the medical device 106. The measurements of the one or more hardware components can include, by non-limiting example, the hardware information 410 as illustrated and described in further detail with respect to FIG. 4.

The subroutine 600 includes a step 604 of measuring one or more software components of the medical device 106. The software components can include, by non-limiting example, the bootloader 317, the operating system 318, the software applications 319, and other authenticity identifiers 400 stored in the medical device memory 312.

The subroutine 600 includes a step 606 of generating hash values 328 pertaining to the measurements of the one or more hardware components and the one or more software components. The hash values 328 are generated using one or more hash functions 332. The generation of hash values 328 using the one or more hash functions 332 is illustrated and described in further detail with respect to FIGS. 3-5.

The subroutine 600 includes a step 608 of storing the generated hash values 328 in one or more PCRs 326. The storage of the hash values 328 in the one or more PCRs 326 allows the security device 320 to compare hash values in an initial PCR with one or more subsequent PCRs as illustrated and described in further detail with respect to FIG. 5.

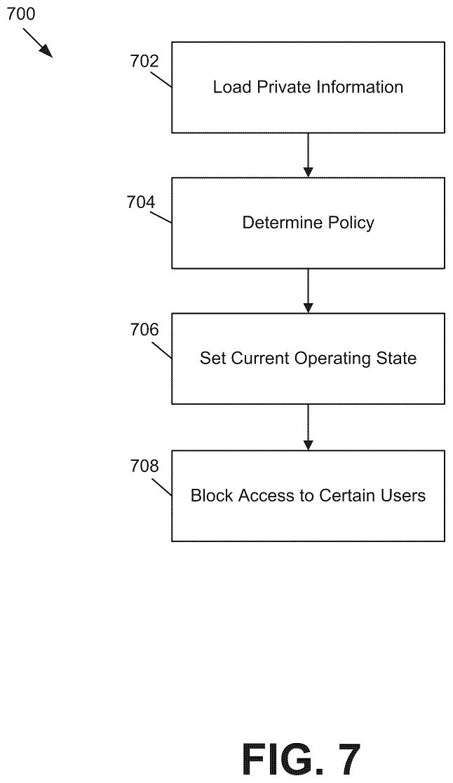

FIG. 7 schematically illustrates an example of a method 700 of determining a policy for protecting private information within the medical device 106 by blocking access to certain users based on the determined policy. The method 700 includes a step 702 of loading private information onto the medical device 106. The private information includes protected health information (PHI). The private information may be obtained by collecting data from a patient. The private information may also be obtained by transferring data to the medical device 106 via the network 108 such as data transferred to the medical device 106 from the EMR system 102.

The method 700 includes a step 704 of determining a policy for protecting the private information. The term “policy” is used herein to describe a set of rules, guidelines, and principles that govern the handling, access, storage, and sharing of data. The policies can change based on a level of confidentiality assigned to the private information. In certain examples, the private information contains protected health information (PHI) such that it requires a well-defined data protection policy. In other examples, information that is stored within the medical device memory 312 or that is accessible via the network 108 may be publicly available such that it does not require a policy for protecting the data. The policy is defined by the manufacturer of the medical device 106. Optionally, in other examples, the policy is defined or modified by a customer who purchases the medical device 106 from the manufacturer such as an organization or facility including, for example, a healthcare facility such as a hospital, a surgical center, a nursing home, a long-term care facility, or similar type of facility.

The method 700 includes a step 706 of determining a current operating state 420 of the medical device 106. For example, step 706 can include determining that the current operating state 420 is in the normal operation mode 422, the service mode 424, or the engineering mode 426. The current operating state 420 determined in step 706 is used to enable the protection of data on the medical device 106 based on the policy determined in step 704.

The method 700 includes a step 708 of blocking access to certain users. Access can be blocked to certain users based on the policy determined in step 704 and the current operating state 420 determined in step 706 on the medical device 106 to protect that the private information. Examples of blocking access to certain users based on the private information, the policy determined to protect the private information, and the current operating state are illustrated and described above with reference to FIGS. 3-7.

In a non-limiting example, and as illustrated and described above with reference to FIG. 4, the security device 320 blocks access to PHI stored within the medical device 106 when the current operating state 420 is in the service mode or the engineering mode. In this example, a policy determined in step 704 protects PHI such that it can be viewed only by valid entities (e.g., a caregiver). To implement this policy, an invalid entity (e.g., a service engineer) is blocked from receiving access to the PHI. Other non-limiting examples of restricting access to certain individuals based on the current operating state 420 of the medical device are described and illustrated above with reference to FIG. 4.

The various embodiments described above are provided by way of illustration only and should not be construed to be limiting in any way. Various modifications can be made to the embodiments described above without departing from the true spirit and scope of the disclosure.

What is claimed is:

1. A security system for integration into a medical device; the security system comprising:
at least one security device processor; and
a security device memory storing instructions which, when executed by the at least one security device processor, cause the at least one security device processor to:
generate one or more initial platform configuration registers corresponding to hardware information and a current operating state of the medical device at a time of manufacture;
generate one or more subsequent platform configuration registers corresponding to the hardware information and the current operating state when the medical device in use after the time of manufacture;
compare the one or more initial platform configuration registers to the one or more subsequent platform configuration registers; and
provide access to the memory when the one or more initial platform configuration registers match the one or more subsequent platform configuration registers.

2. The security system of claim 1, further comprising the medical device, the medical device including:
a medical device processor; and
a memory storing medical device data and firmware configured to retrieve medical device information, the medical device information including the hardware information and the current operating state of the medical device.

3. The security system of claim 2, wherein the memory further stores a bootloader, an operating system, and application code.

4. The security system of claim 3, wherein the application code includes software that is unique to an operation of the medical device.

5. The security system of claim 1, wherein the one or more initial platform configuration registers includes stored hash values corresponding to the hardware information and the current operating state at the time of manufacture; and
wherein the one or more subsequent platform configuration registers include stored hash values corresponding to the hardware information and the current operating state when the medical device is in use after the time of manufacture.

6. The security system of claim 5, wherein the stored hash values are cryptographic.

7. The security system of claim 1, wherein the one or more initial platform configuration registers are compared to the one or more subsequent platform configuration registers using one or more hash comparison functions that are generated by the at least one security device processor and stored within the security device memory.

8. The security system of claim 1, wherein the hardware information includes at least one of a model number, a serial number, a date of manufacture, a memory capacity, and a medical device processor clock speed.

9. The security system of claim 1, wherein the current operating state includes a normal operational mode for collecting and storing medical device data, a service mode for servicing the medical device, and an engineering mode for testing or optimizing the medical device.

10. The security system of claim 1, wherein the memory stores private health information and one or more manufacturer-provided service certificates.

11. The security system of claim 10, wherein the instructions further cause the at least one security device processor to:
prevent access to the private health information when the current operating state is in the service mode or the engineering mode.

12. The security system of claim 10, wherein the instructions further cause the at least one security device processor to:
prevent access to the one or more manufacturer-provided service certificates when the current operating state is in the normal operational mode or the engineering mode.

13. The security system of claim 10, wherein the instructions further cause the at least one security device processor to:
verify an authenticity of application code used to operate the medical device.

14. A method for storing information within a medical device, the method comprising:
integrating a security device into the medical device at a time of manufacture;
generating one or more initial platform configuration registers that correspond to medical device hardware information and a current operating state of the medical device at the time of manufacture; and
storing the one or more initial platform configuration registers within the security device.

15. The method of claim 14, further comprising:
receiving an input requesting access to a memory within the medical device;
generating one or more subsequent platform configuration registers corresponding to the hardware information and the current operating state when the medical device is in use after the time of manufacture;
comparing the one or more initial platform configuration registers to the one or more subsequent platform configuration registers; and
providing access to the memory when the one or more initial platform configuration registers match the one or more subsequent platform configuration registers.

16. The method of claim 14, wherein the one or more initial platform configuration registers includes stored hash values corresponding to the hardware information and the current operating state at the time the medical device is manufactured, wherein the one or more subsequent platform configuration registers include stored hash values corresponding to the hardware information and the current operating state when the medical device is in use after the time of manufacture.

17. The method of claim 14, further comprising generating one or more hash comparison functions configured to provide access to the memory when the one or more initial platform configuration registers match the one or more subsequent platform configuration registers.

18. The method of claim 14, wherein the hardware information includes at least one of a model number, a serial number, a date of manufacture, a memory capacity, and a medical device processor clock speed.

19. A method for accessing information stored within a medical device, the method comprising:
receiving an input requesting access to a memory within the medical device;
generating one or more platform configuration registers corresponding to hardware information and a current operating state when the medical device is in use after a time of manufacture;
comparing the one or more platform configuration registers to one or more initial platform configuration registers stored within a security device memory, wherein the one or more initial platform configuration registers correspond to the hardware information and the current operating state at the time of manufacture; and
providing access to the memory when the one or more initial platform configuration registers match the one or more subsequent platform configuration registers.

20. The method of claim 19, further comprising:
integrating a security device into the medical device at the time of manufacture;
generating the one or more initial platform configuration registers that correspond to the hardware information and the current operating state of the medical device at the time of manufacture; and
storing the one or more initial platform configuration registers within the security device memory.
